(19) United States
(12) Patent Application Publication
(10) Pub. No.: US 2019/0247396 A1
(43) Pub. Date: Aug. 15, 2019

(54) DENTAL TREATMENT

(71) Applicant: KING'S COLLEGE LONDON, Strand, London (GB)

(72) Inventor: Paul Thomas SHARPE, London (GB)

(73) Assignee: KING'S COLLEGE LONDON, Strand, London (GB)

(21) Appl. No.: 16/343,634

(22) PCT Filed: Oct. 20, 2017
(86) PCT No.: PCT/GB2017/053172

§ 371 Date: Apr. 19, 2019

(30) Foreign Application Priority Data
Oct. 21, 2016 (GB) 1617820.4

(51) Int. Cl. A61K 31/506; A61K 9/00; A61K 31/404; A61K 47/42; A61K 31/433; A61K 45/06; A61P 1/02

(52) U.S. Cl. CPC A61K 31/506; A61K 9/0053; A61K 31/404; A61P 1/02; A61K 31/433; A61K 45/06; A61K 47/42

(57) ABSTRACT

A pharmaceutically acceptable small molecule which inhibits GSK-3 activity, such as BIO, CHIR99021 or tideglusib; are used in the repair or regeneration of dentine. Combinations with matrix materials forming dental implants are also described and claimed.

The present invention relates to methods and reagents for use in the regeneration of dentine, in particular for the treatment of conditions associated with dental caries or trauma, as well as to kits for use in these methods.

### BACKGROUND OF THE INVENTION

Dentine is a vital tooth mineral that is produced by highly specialised mesenchymal cells, odontoblasts. It forms a thick layer of porous mineral beneath the enamel that serves as second barrier of defence against infectious agents threatening the inner soft pulp tissue. The dental pulp houses mesenchyme-derived specialised cells, the odontoblasts, that are responsible for dentine secretion throughout life.

When tooth mineral is compromised either following trauma or infection (caries), the inner cellular soft pulp tissue can become exposed to the external environment if the enamel is penetrated and become infected. Metabolic products of microbes and other toxins can diffuse through the dentine tubules and affect the dental pulp cells. In response, resident odontoblasts are stimulated to produce a form of tertiary dentine, reactionary dentine under the area of damage to re-establish the bulk of mineral. The mechanism underlying this stimulation is not fully understood although a role of growth factors sequestered in dentine and released following damage has been suggested.

Clinical repair of tooth damage currently involves the use of mineral aggregates that are used to fill the space in dentine created following removal of decay or trauma. In particular, inorganic hydraulic silicate cements such as mineral trioxide aggregate (MTA) and calcium hydroxide to seal off the exposed dentine tubules from the external environment and help the release of sequestered growth factors.

When the soft inner pulp tissue is exposed, the natural repair process involves the mobilisation of resident mesenchymal stem cells to differentiate into new odontoblast-like cells that replace lost primary odontoblasts and secrete a form of tertiary (reparative) dentine. The reparative dentine produced forms a thin band of dentine (dentine bridge) that serves to protect the pulp from infection by sealing the tooth pulp from the external environment.

In shallow lesions or lesions that have not exposed the dental pulp yet, other factors are thought to play important roles during the repair process. The transforming growth factor β (TGF-β) and the bone morphogenic proteins (BMP) are active components of the dentine extra cellular matrix. Both, TGF-β and BMP, were shown to be released to the dental pulp following tooth damage, regulating cell differentiation, matrix biosynthesis and immune response (Nakashima & Akamine 2005). TGF-β receptors I and II were found to be located on human odontoblasts and an increase of expression was observed after injury, whereas BMP-2 was shown to be important for odontoblast differentiation and dentine secretion in vitro (Sloan et al. 2001, Nakashima et al. 1994). Both pathways act through intracellular proteins called SMADs. TGF-β is transduced to the nuclei via SMADs 2/3, while BMP is transduced to the nuclei via SMADs 1/5/9. In order to block these pathways, the transduction has to be aborted.

Unfortunately, natural reparative dentine formation is insufficient to effectively repair large lesions, such as those involving the loss of dentine after caries removal and hence the artificial mineral aggregates are used to fill the tooth and replace the lost dentine.

It is known that the activation of Wnt/β-cat signalling is an immediate early response to tissue damage and appears to be essential for stimulating the cellular-based repair in all tissues. Recently, the Wnt/β-catenin signalling pathway was shown to be a key regulator of tooth repair in injuries with pulp exposure; it is an activator of resident stem cells and is expressed in early response to injury in several tissues, especially in the dental pulp.

EP-A-202985 reports that substances capable of activating the Wnt signalling pathway can induce differentiation of dental pulp cells into odontoblasts in vitro, and so may be used in the regeneration of dentine. Specific substances recommended in this case are salts such as lithium chloride, which had been reported as having a suppressive role in GSK-3, as well as certain proteins, specifically Norrin and R-spondin2. However, the GSK-3 protein is involved in a wide range of biological pathways, including the Wnt signalling pathway, where it is generally recognised as a suppressor of the Wnt signalling pathway.

However, the treatment of dental conditions in practice involves a number of specific issues. In particular, cost is an issue. Although it may be desirable to regenerate viable dentine to ensure that a tooth maintains its viability long-term, any treatment to achieve this will compete on a cost/benefit basis with conventional low-cost filling procedures. Systemic administration of agents that stimulate dentine repair is not a practical option, because of the dosage levels required to achieve the necessary concentration of drug at the site of the dentine, involving both significant cost and the possibility of side effects.

Local delivery systems, such as by the use of implants is preferable since the active agent is concentrated at the site of use, meaning that low quantities may be used. However, the bioavailability of the agent, as well as factors such as the dissolution rates are crucial, since the agent may be applied once only, by a dentist or dental surgeon, during treatment of a cavity, which is then capped. Salts or proteins as suggested in EP-A-202985 may not have suitable properties in such cases.

Small organic molecules that act in Wnt pathway are known in the art, and some have been shown to have effects on embryonic odontogenesis (M Aurrekoetxea et al. Frontiers in Cell and Developmental Biology, 4, 2016, p1-14), and in the induction of keratinocytes into enamel-secreting ameloblasts (Wang et al., Zhongguo Shengwu Huaxue yu Fenzi Shegwu Xuebao, 30, 8 (2014) p778-786). However, a particular example, 6-bromoindirubin-3′oxime or BIO, has been found to maintain human deciduous tooth dental pulp cells in the undifferentiated state, and reduce proliferation (S. Kawai et al., Shika Yakubutsu Ryoho, 31 (2012), p87-95), suggesting that they may not be useful in the repair or regeneration of dentine. WO2016/109433 describes the use of Wnt stimulator agents, in particular Wnt protein such as human Wnt3A, for enhancing dentine production, which agents are administered in particular in a lipid structure.

### SUMMARY OF THE INVENTION

After confirming that Wnt/β-cat signaling is upregulated following tooth damage (data not shown), the applicants investigated how Wnt signaling agonists may be effectively used to stimulate reparative dentine formation and thus restore lost dentine following caries removal with naturally-generated new dentine.

According to the present invention there is provided a pharmaceutically acceptable small molecule which inhibits GSK-3 activity; for use in the repair or regeneration of dentine.

As used herein, the expression ‘small molecule’ refers to an organic compound, in particular one having a molecular weight of less than 900 daltons, preferably less than 500 daltons.

The molecule is typically produced by synthesis, using conventional chemical methods, although a range of small molecule GSK-3 inhibitors may be derived from marine organisms. These may include the commercially available GSK-3 inhibitor BIO (6-bromoindirubin-3′oxime).

The applicants have found that by targeting specifically GSK-3 within the complex Wnt pathway, molecules can efficiently lead to dentine regeneration in vivo when applied topically, using a suitable delivery system. Thus, they may be beneficially used in the treatment of conditions such as dental caries or dental trauma, to restore tooth vitality.

Furthermore, in cases where the pulp is not penetrated but only the dentine is affected, a dentist will aim to keep as much good dentine as possible before adding the pulp capping reagents. The applicants have found that small molecule GSK3 inhibitors can penetrate dentine and stimulate underlying pulp stem cells to make odontoblast-like cells that make tertiary dentine, or reactionary dentine, providing additional natural healing options in a wider variety of clinical situations. GSK3 is a serine/threonine protein kinase that mediates the addition of phosphate molecules onto serine and threonine amino acid residues. Suitable compounds are inhibitors of GSK-3 activity. This may be because of the role of this enzyme in Wnt signalling, but other effects or pathways may be implicated in the highly efficient regeneration found.

Many small molecule inhibitors of GSK-3 activity are known in the art and have been shown to efficiently upregulate Wnt activity in numerous experimental contexts. These molecules may be ATP-competitive, and so target the ATP binding site of the GSK3 kinase its active conformation. Examples of such inhibitors include aminopyrimidines (such as CHIR98014, CHIR98023, CHIR99021 or TWS119), arylindolemaleimides (such as SB-216763 and SB-41528), thiazoles (such as AR-A014418), indoles (such as AZD-1080), Paullones (such as alsterpaullone, cazpaullone and kenpaullone) and aloisines, as well as some of the marine-derived GSK-3 inhibitors such as BIO (defined above) and other indirubins, or marine alkaloids such as dibromocantharelline, hymenialdesine or meridianins.

Alternatively, the small molecule GSK-3 inhibitors may be non-competitive to ATP. Such molecules include thiadiazolidindiones (such as Tideglusib, TDZD-8, NP00111 or NP03115) or halomethylketones (such as HMK-32), as well as other marine-derived inhibitors such as manazmine A, palinurin or tricantine.

For example, a range of GSK-3 inhibitors suitable for use in the invention are described in US20130028872, the content of which is incorporated herein by reference. In a particular embodiment, the small molecule inhibitor is BIO, of formula (I)

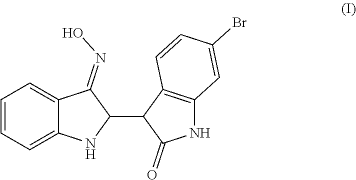 [SMILES: O=C1Nc2cc(Br)ccc2C1C1Nc2ccccc2/C1=N\O]

or a pharmaceutically acceptable salt thereof.

In another embodiment, the small molecule is an aminopyridine or aminopyrimidine, and in particular is an aminopyrimidine as described in WO99/65897, the content of which is incorporated herein by reference.

In summary, these compounds are of formula (II)

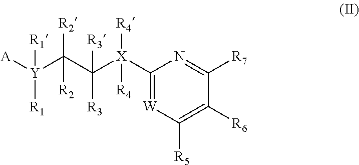

wherein:
W is optionally substituted carbon or nitrogen;
X and Y are independently selected from the group consisting of nitrogen, oxygen, and optionally substituted carbon;
A is optionally substituted aryl or heteroaryl;
R₁, R₂, R₃ and R₄ are independently selected from the group consisting of hydrogen, hydroxyl, and optionally substituted loweralkyl, cycloloweralkyl, alkylaminoalkyl, loweralkoxy, amino, alkylamino, alkylcarbonyl, arylcarbonyl, aralkylcarbonyl, heteroarylcarbonyl, heteroaralkylcarbonyl, aryl and heteroaryl, and R_{1′}, R_{2′}, R_{3′}, and R_{4′} are independently selected from the group consisting of hydrogen, and optionally substituted loweralkyl;
R₆ and R₇ are independently selected from the group consisting of hydrogen, halo, and optionally substituted loweralkyl, cycloalkyl, alkoxy, amino, aminoalkoxy, alkylcarbonylamino, arylcarbonylamino, aralkylcarbonylamino, heteroarylcarbonylamino, heteroaralkylcarbonylamino, cycloimido, heterocycloimido, amidino, cycloamidino, heterocycloamidino, guanidinyl, aryl, biaryl, heteroaryl, heterobiaryl, heterocycloalkyl, and arylsulfonamido;
R₆ is selected from the group consisting of hydrogen, hydroxy, halo, carboxyl, nitro, amino, amido, amidino, imido, cyano, and substituted or unsubstituted loweralkyl, loweralkoxy, alkylcarbonyl, arylcarbonyl, aralkylcarbonyl, heteroarylcarbonyl, heteroaralkylcarbonyl, alkylcarbonyloxy, arylcarbonyloxy, aralkylcarbonyloxy, heteroarylcarbonyloxy, heteroaralkylcarbonyloxy, alkylaminocarbonyloxy, arylaminocarbonyloxy, formyl, loweralkylcarbonyl, loweralkoxycarbonyl, aminocarbonyl, aminoaryl, alkylsulfonyl, sulfonamido, aminoalkoxy, alkylamino, heteroarylamino, alkylcarbonylamino, alkylaminocarbonylamino, arylaminocarbonylamino, aralkylcarbonylamino, heteroarylcarbonylamino, arylcarbonylamino, heteroarylcarbonylamino cycloamido, cyclothioamido, cycloamidino, heterocycloamidino, cycloimido, heterocycloimido, guanidinyl, aryl, heteroaryl, heterocyclo, heterocycloalkyl, arylsulfonyl and arylsulfonamido; and the pharmaceutically acceptable salts thereof.

In this context, the expression “optionally substituted” refers to the replacement of hydrogen with a monovalent or divalent radical. Suitable substitution groups include, for example, hydroxyl, nitro, amino, imino, cyano, halo, thio, thioamido, amidino, imidino, oxo, oxamidino, methoxamidino, imidino, guanidino, sulfonamido, carboxyl, formyl, loweralkyl, haloloweralkyl, loweralkoxy, haloloweralkoxy, loweralkoxyalkyl, alkylcarbonyl, arylcarbonyl, aralkylcarbonyl, heteroarylcarbonyl, heteroaralkylcarbonyl, alkylthio, aminoalkyl, cyanoalkyl, and the like.

The substitution group can itself be substituted. The group substituted onto the substitution group can be carboxyl, halo; nitro, amino, cyano, hydroxyl, loweralkyl, loweralkoxy, aminocarbonyl, —SR, thioamido, —SO₃H, —SO₂R or cycloalkyl, where R is typically hydrogen, hydroxyl or loweralkyl.

When the substituted substituent includes a straight chain group, the substitution can occur either within the chain (e. g., 2-hydroxypropyl, 2-aminobutyl, and the like) or at the chain terminus (e. g., 2-hydroxyethyl, 3-cyanopropyl, and the like). Substituted substitutents can be straight chain, branched or cyclic arrangements of covalently bonded carbon or heteroatoms.

“Loweralkyl” as used herein refers to branched or straight chain alkyl groups comprising one to ten carbon atoms that are unsubstituted or substituted, e. g., with one or more halogen, hydroxyl or other groups, including, e. g., methyl, ethyl, propyl, isopropyl, n-butyl, t-butyl, neopentyl, trifluoromethyl, pentafluoroethyl and the like.

Suitable embodiments of formula (II) are as described in WO99/658897, the content of which is incorporated herein by reference. In particular, the compound of formula (II) is a pyrimidine of formula (IIA)

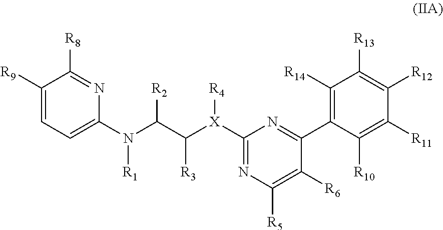

where R₁-R₆ are as defined above,
R8 and R9 are independently selected from the group consisting of hydrogen, nitro, amino, cyano, halo, thioamido, amidino, oxamidino, alkoxyamidino, imidino, guanidinyl, sulfonamido, carboxyl, formyl, loweralkyl, haloloweralkyl, loweralkoxy, haloloweralkoxy, loweralkoxyalkyl, loweralkylaminoloweralkoxy, loweralkylcarbonyl, loweraralkylcarbonyl, lowerheteroaralkylcarbonyl, alkylthio, aryl and, aralkyl; R₁₀, R₁₁, R₁₂, R₁₃ and R₁₄ are independently selected from the group consisting of hydrogen, nitro, amino, cyano, halo, thioamido, carboxyl, hydroxy, and optionally substituted loweralkyl, loweralkoxy, loweralkoxyalkyl, haloloweralkyl, haloloweralkoxy, aminoalkyl, alkylamino, alkylthio, alkylcarbonylamino, aralkylcarbonylamino, heteroaralkylcarbonylamino, arylcarbonylamino, heteroarylcarbonylamino aminocarbonyl, loweralkylaminocarbonyl, aminoaralkyl,loweralkylaminoalkyl, aryl, heteroaryl, cycloheteroalkyl, aralkyl, alkylcarbonyloxy, arylcarbonyloxy, aralkylcarbonyloxy, arylcarbonyloxyalkyl, alkylcarbonyloxyalkyl, heteroarylcarbonyloxyalkyl, aralkycarbonyloxyalkyl, and heteroaralkcarbonyloxyalkyl; or a pharmaceutically acceptable salt thereof.

A commercially available compound of formula (IIA) is CHIR₉₉₀₂₁ of formula (IIB)

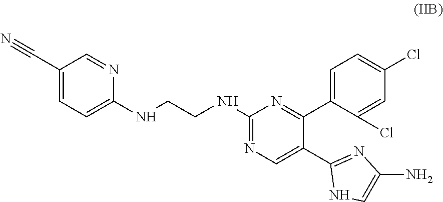 [SMILES: N#Cc1ccc(NCCNc2ncc(-c3nc(N)c[nH]3)c(-c3ccc(Cl)cc3Cl)n2)nc1]

and this forms a particular embodiment of the invention.

In yet another embodiment, the inhibitor is a thiazolidinone as described in WO2005/097117, the content of which is incorporated herein by reference. In particular, the inhibitor is a compound of formula (III)

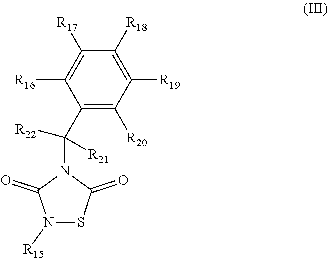

wherein R₁₅ is an organic group having at least 8 atoms selected from C or O, which is not linked directly to the N through a —C(0)- and comprising at least an aromatic ring; and R₁₆, R₁₇, R₁₈, R₁₉, R₂₀, R₂₁ and R₂₂ are independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, —COR₂₃, —C(O)OR₂₂, —C(O)NR₂₂R₂₄—C—NR₂₃, —CN, —OR₂₂, —OC(O)R₂₂, —S(O)ₜ—R₂₂, —NR₂₂R₂₄, —NR₂₂C(O)R₂₄, —NO₂, —N—CR₂₃R₂₄ or halogen, t is 0, 1, 2 or 3,
R₂₂ and R₂₄ are each independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, substituted or unsubstituted alkoxy, substituted or unsubstituted aryloxy, halogen;
wherein R₂₁ and R₂₂ together can form a group ═O, and wherein any pair R₂₁ R₁₆, R₁₆ R₁₇, R₁₇ R₁₈, R₁₈ R₁₉, R₁₉ R₂₀, R₂₀ R₂₂, or R₂₃ R₂₄ can form together a cyclic substituent;
or a pharmaceutically acceptable salt thereof.

In particular R₁ is an aromatic group such a naphthyl.

Suitable examples of compounds of formula (III) are described in WO2005/097117. In particular, the compound of formula (III) is Tideglusib of formula (IIIA)

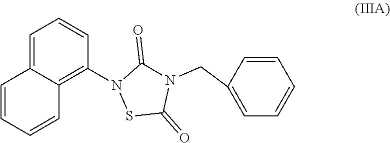 [SMILES: O=c1sn(-c2cccc3ccccc23)c(=O)n1Cc1ccccc1]

or a pharmaceutically acceptable salt thereof.

In yet another embodiment, the small molecule GSK3 inhibitor is a thiazole derivative, for example as described ion WO03/089419, the content of which is incorporated herein by reference. Specifically, such compounds may be of formula(IV)

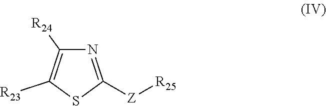

wherein: Z is NHCONH, NHCO, or NH;
R₂₃ is nitro or COR₂₆;
R₂₄ is hydrogen or NH₂;
R₂₅ is C₁₋₆alkyl or C₀₋₆alkylaryl wherein C₀₋₆alkylaryl may be substituted by one or more groups R₂₂;
R₂₆ is C₁₋₆alkyl; and
R₂₇ is independently selected from halo, OR₂₈ and C₁₋₆alkyl; and R₂₈ is C₁₋₆alkyl.

In particular, the compound of formula (IV) is a urea, where Z is a group NHCONH.

Suitably Rn is a benzyl group which is optionally substituted by one or more R₂₇ groups.

Suitably R₂₃ is nitro and R₂₄ is hydrogen.

A particular example of a compound of formula (IV) is AR-A014418 or N-(4-methyoxybenzyl)-N′—(5-nitro-1,3-thiazol-2-yl) urea of formula (IVA)

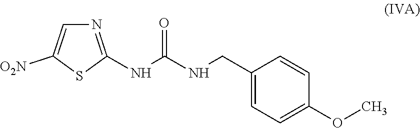 [SMILES: COc1ccc(CNC(=O)Nc2ncc([N+](=O)[O-])s2)cc1]

According to a further aspect of the invention, there is provided a method repairing or regenerating dentine; which comprises administering to a patient in need thereof, an effective amount of a small molecule which inhibits GSK-3 as described above.

In particular, the small molecule is administered topically, directly to an area comprising exposed dentine, for example a cavity in a tooth occurring as a result of dental caries or trauma, or as exposed following dental drilling.

It is suitably administered either alone or in the form of a pharmaceutically acceptable composition, in which it is combined with a pharmaceutically acceptable carrier. In particular, the pharmaceutically acceptable composition is a hydrophilic composition. In particular, the composition will not comprise a lipid or liposomes. This ensures that the small molecule will be able to readily access the exposed dentine. In particular, the pharmaceutically acceptable carrier is water or an aqueous buffer solution such as phosphate buffered saline. If necessary, a solubilising agent such as dimethylsulphoxide (DMSO) may be used to facilitate the dissolution of the small molecule.

For topical application, it may be suitable to form a thickened or paste-like composition comprising the small molecule. In that case, suitable excipients may include thickeners, nanopastes, nanoneedles or hydrogels.

In a particular embodiment, the small molecule or composition comprising it, is administered using a dental implant, comprising a matrix material which carries the small molecule.

Thus in a further aspect, the invention provides a combination of a matrix material suitable for use in a dental implant, and a pharmaceutically acceptable small molecule which inhibits GSK-3 activity.

The matrix material is suitably porous, for example in the form of a sponge such as a collagen or gelatine sponge, so that the small molecule may be impregnated into the matrix material. The matrix material may be cut and shaped to fill the cavity being treated.

The matrix material suitably comprises a biodegradable material. The degradation rate of the biodegradable matrix material is suitably such that it degrades at substantially the same rates as new dentine forms, so that no unwanted voids are formed during the repair process. In particular, the matrix material is a collagen or gelatine sponge and in particular a collagen sponge. Collagen is a naturally occurring protein found in a wide variety of animal species.

A particularly convenient source of collagen is fish collagen.

When used in this way, as the dentine grows, as a result of stimulation from the small molecule, it may fill the space left as a result of the degradation of the implant material.

The small molecule is suitably in solution in a pharmaceutically acceptable carrier prior to addition to the matrix material.

Alternatively however, the small molecule may be administered to the surface of a matrix material. In this case, the small molecule may be in the form of a solid or liquid pharmaceutically acceptable composition, which includes a conventional pharmaceutically acceptable carrier.

The concentration and amount of the small molecule present on the matrix will vary depending upon factors such as the nature of the small molecule, the size of the cavity and the size and age of the patient being treated. However, local delivery of the small molecule directly to the site of use in this manner ensures efficient use of the agent. Provided the concentrations selected is not inherently cytotoxic (and this may be tested using routine methods as described for example hereinafter), it may be preferable to use the highest concentration possible to ensure that sufficient stimulation is achieved with just a single application.

If necessary however, additional doses of the small molecule may be administered subsequently by a dental surgeon, in particular if temporary capping means are used after the first administration.

Typically, concentrations of solutions of small molecule in the range of from 0.001 nM to 1 mM, depending upon the factors described above, would be applied to the matrix material. Thus, dosages of the small molecule administered will also vary depending upon factors such as the size of the cavity, the health of the patient, the nature of the condition being treated etc. in accordance with normal clinical practice.

Typically, a dosage in the range of from 1 μg-50 mg/Kg such as from 1-50 μg/Kg but in particular from 1-50 mg/Kg, for instance from 2-20 mg/Kg, such as from 5-15 mg/Kg would be expected to produce a suitable effect.

The GSK3 inhibitor may be administered alone or in combination with other active agents such as antibiotics, which may be useful in particular in cases where infection is present, such as in cases of deep caries. The additional agent may be administered to the matrix material either together or separately from the GSK3 inhibitor. The presence of agents such as antibiotics would not affect the repair. Suitable antibiotics will include those commonly used in dental treatments such as Amoxicillin and others.

Again, the amount of antibiotic or other active substance administered will depend upon factors such as the nature of the substance, the condition being treated, the nature of the patient and so will be determined by the clinician. Typically however, the antibiotic will be administered in an amount of from 1 μg-50 mg/Kg such as from 1-50 μg/Kg but in particular from 1-50 mg/Kg, for instance from 2-20 mg/Kg, such as from 5-15 mg/Kg.

In addition, in view of the fact that the presence of TGF-β and BMP has been demonstrated to modulate dentine structure, it may be desirable also to include an agonist of either TGF-β or BMP in the combination to supplement the latent protein and so ensure good tubular organisation of reparative or reactionary dentine. Agonists of these proteins may comprise the proteins themselves, or in particular other small molecules.

Many agonists of TGF-β and BMP are known in the art. For example, agonists of TGF-β are described in. U.S. Pat. Nos. 8,097,645 and 8,410,138.

Agonists of EMP are described for example by Vrigens et al., PLoS one, March 2013, Volume 8, Issue 3, e59045.

In that case, the dosage administered will again depend. upon the agent used.

Once inserted into a cavity where dentine is exposed, the implant is suitably held in place and isolated from the environment by means of a sealant, such as conventional dental cap, crown or ionomer.

Combinations of the invention may be supplied in the form of a kit, for use in a dental practice and these provide yet a further aspect of the invention. In a particular embodiment, the kit comprises matrix material and a small molecule GSK3 inhibitor packaged separately, for example in a two-part container. Each package will suitably be sterile. The dentist may then shape the matrix material to fit the cavity either before or after administration of the small molecule to it. The small molecule will suitably be in the form of a pharmaceutical composition, as described above. It may be applied directly to the surface of the matrix, or if necessary, dissolved in a sterile liquid carrier before application to the matrix. Where the matrix is a porous material, it is suitably soaked in a solution of the small molecule, just prior to application. Kits may further comprise additional active substances, including antibiotics, TGF-β agonist or BMP agonists as described above.

As described hereinafter, the applicants tested the ability of three small molecule GSK3 inhibitors, two ATP competitive molecules, BIO (6-bromoindirubin-3′-oxime) and CHIR₉₉₀₂₁(6-[[2-[[4-(2,4-Dichlorophenyl)-5-(5-methyl-1H-imidazol-2-yl)-2pyrimidinyl]amino]ethyl]amino]-3-pyridinecarbonitrile) and one non-ATP competitive molecule, Tideglusib (4-Benzyl-2—(naphthalen-1-yl)-[1,2,4]thiadiazolidine-3,5-dione) to stimulate tertiary dentine following experimentally induced pulp exposure.

As a delivery vehicle, a commercially-available, clinically approved collagen sponge was used. All molecules promoted the successful regeneration of dentine in a tooth cavity.

Modern dental practice for carious lesions aims to remove decay and restore tooth structure by using mineral aggregate filling materials. Preservation of undamaged dentine forms an integral part of this practice since maintenance of as much of the natural mineral as possible is deemed important for tooth vitality. Mineral aggregates such as MTA and Biodentine are reported to aid the formation of tertiary dentine, although the deposition of this dentine is not at the sites of damage but rather internal in the pulp space.

As described hereinafter, the applicants found no effects of MTA on Wnt signalling activity, and although it may be acting via other pathways it seems likely that any positive action on mineralisation is as result of providing mineral ions.

The method devised by the applicants used an already clinically-approved biomaterial (such as collagen sponge —Kolspon®) as a delivery vehicle for small molecule GSK-3 inhibitors (Wnt agonists). Wnt/pcatenin signalling has emerged as a major target in tissue regeneration and repair and this pathway activity can be stimulated in a number of different ways. The small molecule GSK-3 inhibitors provide a simple, cost-effective method that is supported by substantial existing experimental data and clinical use. Both BIO and CHIR₉₉₀₂₁ have been extensively used experimentally to elevate Wnt activity by inhibiting GSK-3, while Tideglusib is in clinical trials for systemic use in the treatment of neurological disorders include Alzheimers disease. Since upregulated Wnt activity in response to damage is an immediate early response, it is important to achieve rapid release of small molecule GSK3 inhibitors, and this was achieved using a collagen sponge.

All three GSK3 inhibitors tested showed significantly increased mineralisation at the site of damage compared to the use of the sponge alone or MTA. More significantly the localization of the reparative dentine formed indicated that the mineral replaced the biodegradable sponge and restored the cavity in the dentine made by the burr. With MTA the cavity remains permanently filled with mineral aggregate and this non-degradable material can only affect reparative dentine formation on the pulp chamber aspect.

Small molecule GSK-3 inhibitors, (which may act as Wnt signalling agonists), delivered via a biodegradable collagen sponge provide an effective repair of experimentally-induced deep dental lesions by promotion of reparative dentine formation. The simplicity of this approach makes it ideally translatable into a clinical dental product for treatments requiring dentine restoration and pulp protection that are currently treated with non-organic cements.

The invention will now be particularly described by way of example with reference to the accompanying diagrammatic drawings.

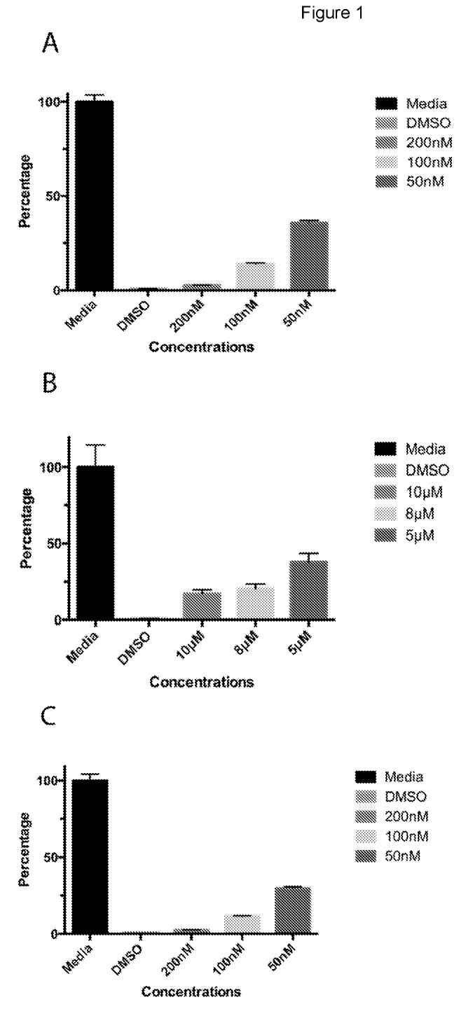

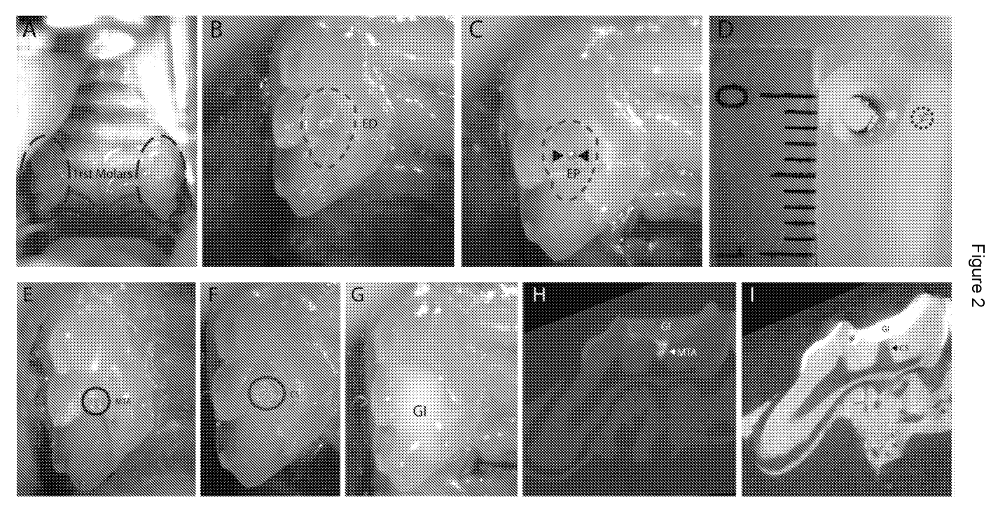

FIG. 1. Drug Titration and Agonist Activation of the Wnt Pathway

MTT cytotoxity assay for (A) BIO, (B) CHIR₉₉₀₂₁, and (C) Tideglusib. (D) Axin2 qPCR for the In vitro assay with the 171IA cell line shows that when 50 nM BIO, 5pm CHIR, and 50 nM

Tideglusib are in the sponge, Wnt activity increases after 30 minutes of incubation and remains elevated. This elevation is not seen when just media or collagen sponge without the drug are incubated with the cells. (E) Axin2 qPCR for dental pulp cells collected either without injury or after one day of injury and capping with the conditions. BIO, CHIR and Tideglusib shows significant upregulation of Wnt activity when compared with control, MTA or collagen sponge. *P=0.0365, ****P<0.0001.

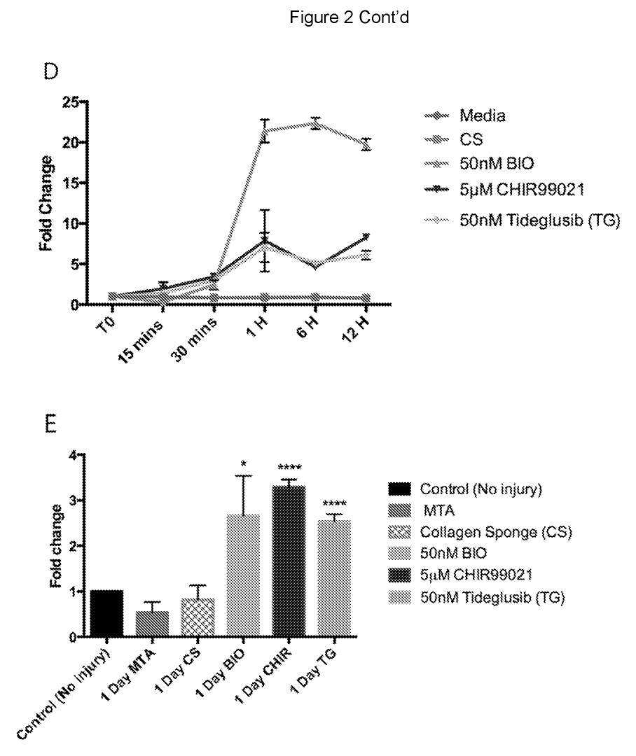

FIG. 2. Injury and Direct Tooth Capping

(A) photograph of upper first molars. (B) A ¼ carbide bur cuts the tooth exposing the dentine until the roof of the pulp chamber (red dashed line). (C) Using a needle the dental pulp is exposed indicated by the arrowheads. (D) The collagen sponge is soaked in drug and a small piece of it, indicated by the black dashed line, is removed for the direct capping. (E)

The injury capped with MTA. (F) The sponge piece condensed inside the exposed pulp area. (G) The tooth is then sealed with glass ionomer until the date of collection. (H) MicroCT image right after capping showing the close contact of MTA (RO area indicated by arrow) with the dental pulp and the glass ionomer sealing. (I) MicroCT image right after capping showing the close contact of the collagen sponge (RL area indicated by arrow) with the dental pulp and the glass ionomer sealing. ED, exposed dentine; EP, exposed pulp; CS, collagen sponge; GI, glass ionomer; RO, radiopaque; RL, radiolucent.

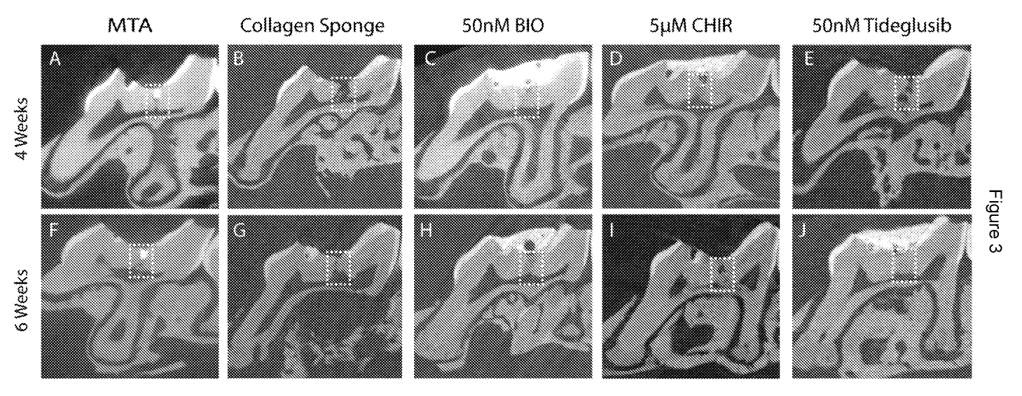

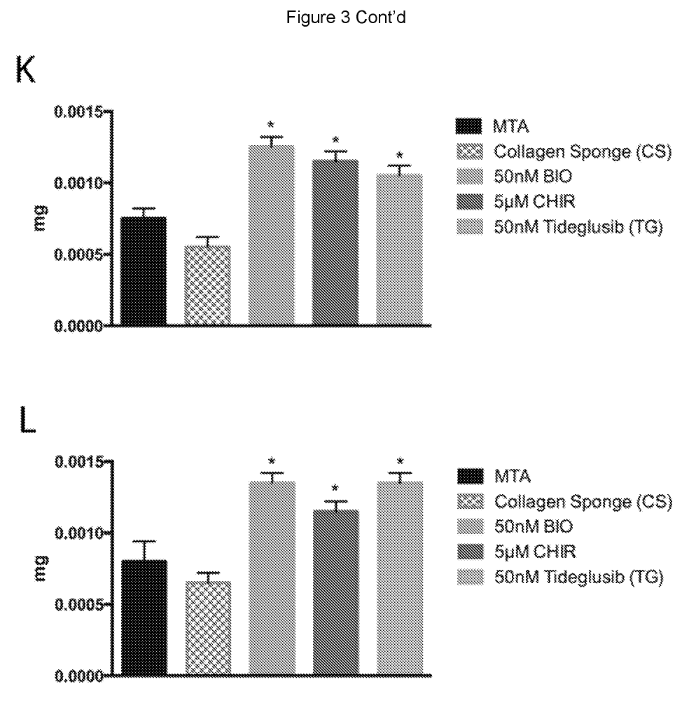

FIG. 3. MicroCT Analysis of Mineral Deposition

(A) MTA repair after 4 weeks, note the material (strong RO area at the injury site) at the injury site. (B) Collagen sponge repair after 4 weeks, spaced dentine formation at the injury site. (C) BIO, (D) CHIR, and (E) Tideglusib repairs show mature mineral at the injury site after 4 weeks. (F) MTA repair after 6 weeks still shows material at the injury site (strong RO area at the injury site). (G) Collagen sponge treatment shows injury mildly repaired. (H) BIO and (I) CHIR repair after 6 weeks displays injury site filled with mature dentine. (J) Tideglusib repair after 6 weeks shows mature reparative dentine formed at the injury site almost at the same Radiopacity as the primary/secondary dentine. No external material is seen at the injury site after repair when teeth were treated with signalling modulators in collagen sponge. (K, L) 4 and 6 weeks, respectively, Mineral formation analysis at the injury site shows that teeth treated with small molecules form more mineral than when treated either with collagen sponge or MTA. 4 weeks BIO *P=0.0101, 4 weeks CHIR *P=0.0136, 4 weeks Tideglusib *P=0.0194; 6 weeks BIO *P=0.0101, 6 weeks CHIR *P=0.0194, 6 weeks Tideglusib *P=0.0101.

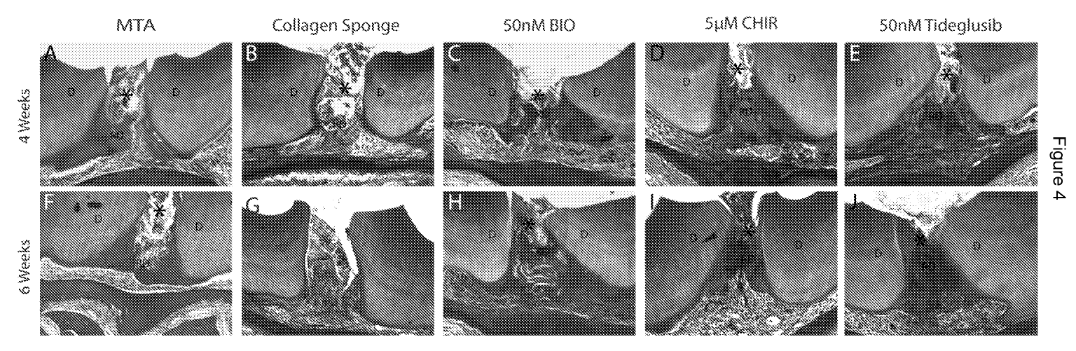

FIG. 4. Histology of Reparative Dentine Formation And Pulp Vitality

(A) 4 weeks MTA repair shows dentine formed underneath where the material was placed. (B) Collagen sponge shows sparse dentine formation in the dental pulp. (C) BIO, (D) CHIR, and (E) Tideglusib repairs show dense dentine formation at the injury site with vital pulp after 4 weeks. (F) 6 weeks MTA repair shows dentine formed underneath where the material was placed. (G) Collagen sponge repair shows little and immature dentine formed at the injury site after 6 weeks. (H) BIO treatment shows new mature dentine formed where the sponge was placed filling the injury site. (I) CHIR treatment shows mature new mature dentine formed where the sponge was placed filling the injury site. (J) Tideglusib treatment shows complete repair with vital dental pulp after 6 weeks.

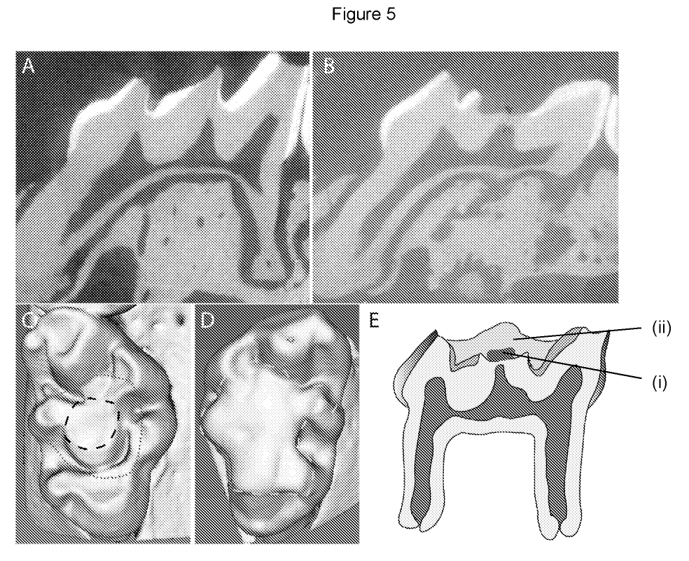

FIG. 5. Non-Exposed Pulp Injury Model. (A) μCT of sound mouse upper first molar displaying the three cusps and pulp horns. (B) Linear measuring of damage on mouse molar without pulp exposure reveals a dentine band of 0.08 mm between pulp horn and floor of the cavity. (C) 3D reconstruction of damage reveals no pulp exposure; Dotted line indicates the area where the dentine was cut, and the dashed line, the area where the capping material is placed. (D) 3D reconstruction of sealed tooth shows glass ionomer sealing the damage (dashed line). (E) Schematic of damage model ((i)—capping material, (ii)—sealing material).

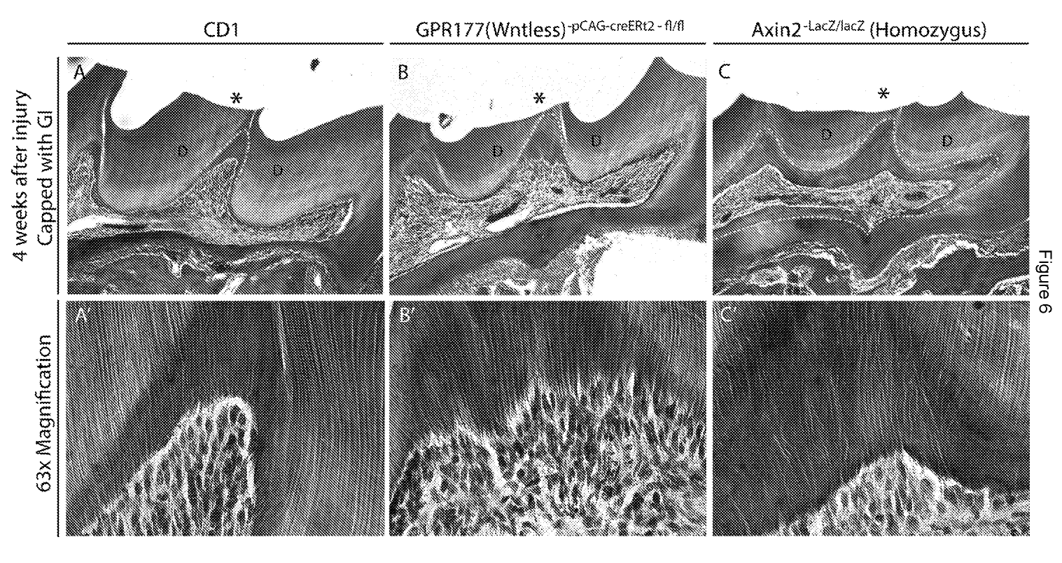

FIG. 6. Masson trichrome staining of wild type (CD1) and mutant mice, 4 weeks after injury without pulp exposure with glass ionomer sealing. (A, B; A′, B′) CD1 and Wntless mice display reactionary dentine repair with normal tubular structure. (C, C′) Axin2 Homozygus mouse molars display increased reactionary dentine secretion within the pulp chamber with irregular tubular structure. Dotted line outlines secreted reactionary dentine. *, Damage site.

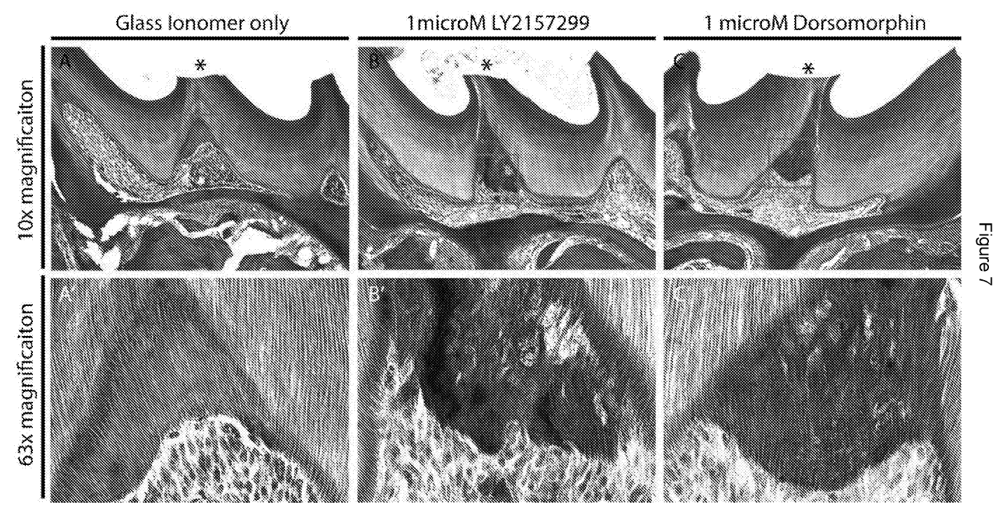

FIG. 7. 4 weeks of repair in wild type mouse molars injured without pulp exposure and capped with TGF-β and BMP inhibitors and control (GI only). (A, A′) Mouse molars capped with glass ionomer only show normal, tubular reactionary dentine secretion. (B, B′) Molars capped with collagen sponge soaked in LY2157299 show atypical globular dentine without tubular reactionary dentine features. (C, C′) Molars capped with collagen sponge soaked in Dorsomorphin show tubular reactionary dentine discontinued by globular dentine. (Squares delineate the magnified area). *, Damage site.

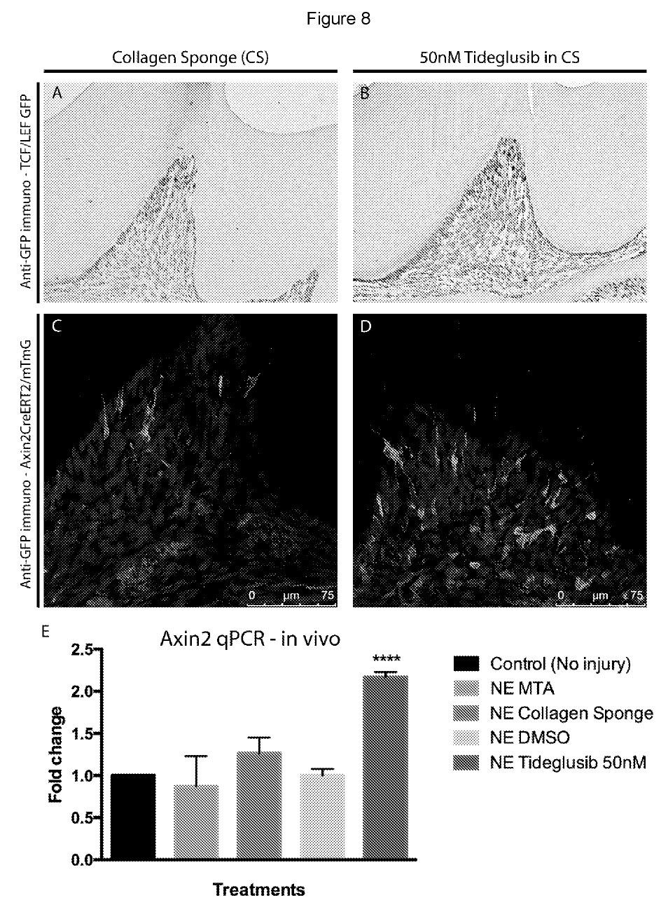

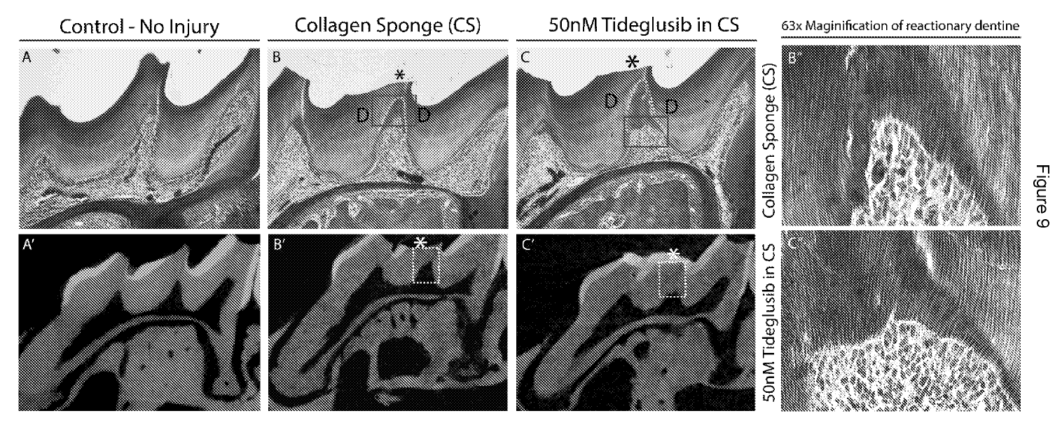

FIG. 8. Wnt responsive cells 1 day after injury in reactionary dentine repair. Immunohistochemistry against GFP reveals increase of TCF/LEF+cells and Axin2+ cells right under the injury in teeth capped with GSK-3 inhibitor (Tideglusib -TG) in collagen sponge (B, D) when compared with collagen sponge alone (A, C). (E) Axin2 Q-PCR for dental pulp collected one day after dentine was injured without exposing the dental pulp and treated with different capping. Tideglusib shows significant upregulation of Wnt activity in comparison to the positive (MTA) and negatives controls (No damage, CS, and DMSO). ****p<0.0001

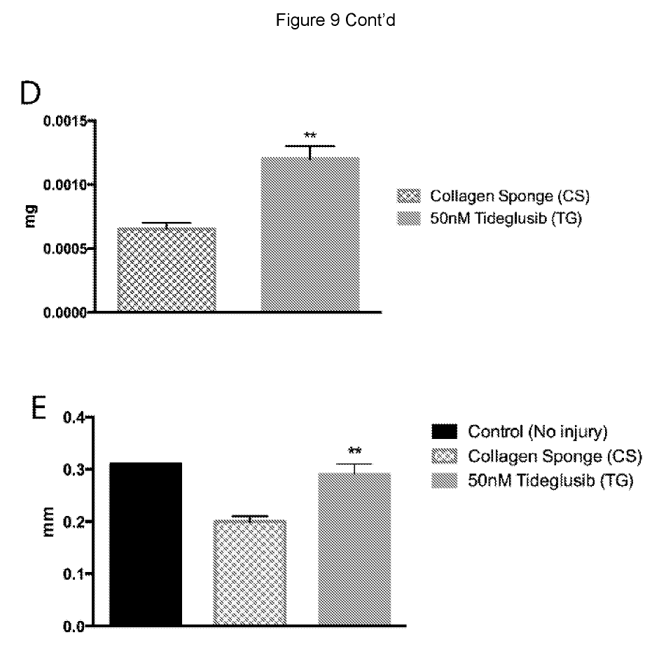

FIG. 9. 4 weeks of repair in wild type (CD1) mouse molars capped with GSK-3 inhibitor in vehicle and controls. (A, A′) Mouse molar without injury shows where injury was created (Dotted line) and the shape of the middle pulp horn without injury. (B, C) Dotted line delineates middle pulp horn, showing the reactionary dentine formed when capping molars with collagen sponge only or 50 nM Tideglusib respectively, showing larger reactionary dentine secretion when GSK-3 inhibitor is used. This finding was confirmed by pCT (B′, C′). (B″, C″) Magnification of squares on images B and C reveal tubular reactionary dentine in both cappings. (E) pCT linear measurement confirms that the distance from the top of the middle pulp horn to the point where the dentine was cut was significantly smaller when molars were capper with collagen sponge only comparison to molars without injury or capped with 50 nM Tideglusib. These results were reflected by the high mineral content in the injured area (D). D, dentine; *, damaged area. (D)**P=0.001; (E)**P=0.0022.

### EXAMPLE 1

### Effective Concentrations and Cytotoxicity Testing

171A4 mouse dental pulp cells were incubated with a range of concentrations of the three small molecule GSK inhibitors,

BIO, CHIR₉₉₀₂₁ and Tideglusib, and cytotoxicity analysed with the MTT assay after 24h in culture. Specifically, 171A4 mouse dental pulp cells were plated in 96 well plates at 20,000 cells/cm² and incubated (37° C., 5% CO₂/95% air, 100% humidity) for 24 hours using standard culture medium. Thereafter, the medium was replaced with conditioned (drugs +media) and control media for another 24hrs. (10 μl of drug in DMSO+90 μl of medium resulting in the following concentrations BIO: 200, 100, 50 nM; CHIR99021: 10, 8, 5 μM; Tideglusib: 200, 100, 50nM). For cell metabolic activity, MTT (3-(4,5-Dimethylthiazol-2-yl)-2,5- diphenyltetrazolium bromide, Sigma) was added after 24hrs. The resulting formazan product was dissolved in 200p1 dimethyl sulfoxide (DMSO, Sigma). A colorimetric plate reader (Thermo Multiskan Ascent 354 microplate reader) was used to read the absorbance at 540 nm with background subtraction at 630 nm.

The results are shown in FIGS. 1A-C. The highest concentration of inhibitor that was not cytotoxic was used in separate assays with the same cells and levels of Axin2 measured by qPCR in the first 24 hours of culture.

In vitro drug release from Kolspon sponge was tested. 171A4 cells were plated in 24-well plates and incubated (37° C., 5% CO2/95% air, 100% humidity) for 24 h using standard culture medium. Falcon™ cell culture inserts for use with 24-well plates (3pm pore size) were placed in the wells carrying 96 mm² Kolspon cubes either dry or soaked in 30 μl of the drug optimal concentration for 15 and 30 minutes, 1, 6, and 12 hours. The cells were collected with TRIzol and stored at −20° C.

RNA was extracted from the cells using TRIzol (Thermo Fisher Scientific) as recommended by the manufacturer. RNA was quantified using Nanodrop and reverse transcribed into cDNA. Beta-actin was used as housekeeping gene (Forward-GGCTGTATTCCCCTCCATCG (SEQ ID NO 1), Reverse-CCAGTTGGTAACAATGCCTGT) (SEQ ID NO 2) and Axin2 for Wnt activity (Forward-TGACTCTCCTTCCAGATCCCA (SEQ ID NO 3), Reverse- TGCCCACACTAGGCTGACA (SEQ ID NO 4).

Increased Axin2 expression was observed after 30 mins reaching a maximum after 1 hr (FIG. 1D). BIO induction of Axin2 expression was 4× greater than both CHIR₉₉₀₂₁ and Tideglusib, each of which showed similar levels of induction (FIG. 1D).

### EXAMPLE 2

### Testing Induction of Axin2 in vivo in mice

To test the induction of Axin2 in vivo, an injury model was developed. Mice were anaesthetized with a solution of Hypnorm (Fentanyl/fluanisone—VetaPharma Ltd.), water and Hypnovel (Midazolam—Roche) in the ratio 1:2:1 at 10 ml/kg by an intraperitoneal injection. Experimental tooth damage was created by drilling and making 0.13 mm holes in mouse maxillary first molars to expose the pulp. A rounded carbide bur FG ¼ coupled to a high speed hand piece was used to access the dentine. Once the bur cut exposed the dentine, a 30G needle was used to penetrate the pulp.

In order to protect the pulp from external contamination and stimulate dentine repair, the injury was capped either with ProRoot Mineral Trioxide Aggregate (MTA) (Maillfer Dentsply), or Kolspon (Fish Collage Type 1—Eucare Ltd) alone, or in association with 50 nM BIO (SIGMA), 5 μM CHIR₉₉₀₂₁ (SIGMA), or 50 nM Tideglusib (SIGMA) dissolved and diluted in DMSO, in contact with the pulp. Pieces of Kolspon were cut to size and soaked in solutions of the three inhibitors before being physically placed into the holes, in contact with the pulp.

A glass ionomer cement was used to cover the sponge and protect the tooth (FIG. 2G). Specifically, a layer of 3M Ketac-Cem Radiopaque was used as a capping material to seal the injured site. The injury was performed on the two upper first molars. Post-op the mice were given Vetergesic (Buprenorphine—Ceva) at the rate of 0.3 mg/kg intraperitonially as analgesic. The animals were sacrificed after 1 day.

Treated teeth were removed after 24 h along with controls consisting of untreated teeth, MTA only and collagen sponge with no inhibitor.

Pulp collection P21 mice had their superior first molars drilled according to the drilling protocol and tooth pulp tissue collected. Molars were extracted using a 21G needle as an elevator to lift them from the alveolar bone and kept in ice cold PBS. Using a 23 scalpel blade the molars were separated at the crown-root junction, so that the pulp chamber could be visualized. Using a 0.6 mm straight tip tweezer the pulp was gently scraped from the pulp chamber and the root canal. The pulp was then placed into cold Sigma RNAlater and stored at −80° C.

The extracted cells were tested for expression of Axin2 by qPCR as described in Example 1 (FIG. 1E). Expression of Axin2 was 3× higher in inhibitor treated pulp cells when compared to controls (FIG. 1E). Significantly MTA showed no effect on Axin2 expression suggesting current protocols do not activate Wnt signalling.

This shows that this experimental model of tooth damage and pulp exposure provides a way of delivering small molecules that were able to affect pulp cell gene expression in a reproducible way.

### EXAMPLE 3

### Reparative Dentine Formation

The model described in Example 2 was then used to examine the effect on the formation of reparative dentine. Molars were drilled and sponges inserted and left as described in Example 2, this time for 4-6 weeks before the mice were sacrificed. Micro-computed tomographic (μCT) scanning was used to visualise and quantify mineral deposition at the drill site. Mice upper molars were fixed with PFA 4% overnight and scanned using a Bruker Skyscan1272 micro-CT scanner. Microview software programme (GE) was used for visualization and analysis. Two dimensional (2D) images were obtained from micro-CT cross-sectional images of superior first molar internal part, to evaluate the drilling and mineral formation. To assay tissue mineral content a ROI of X=0.2 mm, Y=0.4 mm, and Z=0.2 mm was set as standard for all the samples and mineral analysis performed. T filled with mineral=0.0017 mg.

Analysis at both 4 and 6 weeks revealed increased mineralisation with all three agonists compared to controls (FIG. 3 KL). These increases were statistically significant at 4 and 6 weeks. Overall the mineralisation with the inhibitors was on average 2× higher than in the sponge alone control and 1.7× higher than with MTA treatment.

After 4 weeks decalcification in 19% EDTA the teeth were embedded in wax blocks and sectioned using 0.8 μm thickness. Sections were stained using Masson's Trichrome to reveal new dentine formation. The sections confirmed the μCT data showing that teeth treated with GSK inhibitors had reparative dentine was formed at the injury site than with collagen sponge or MTA (FIG. 4). Moreover, the new dentine formed presented as dense dentine localised centrally to the injury site, revealing no remaining collagen sponge where the dentine was formed. Interestingly, by 6 weeks of treatment, the reparative dentine secreted when teeth were treated with BIO, CHIR, and Tideglusib filled the whole injury site from occlusal to pulp chamber roof (FIG. 4H-J). Most importantly, dental pulp remained vital (FIG. 4 H-J).

### EXAMPLE 4

### Effect of Wnt Signaling Modulation on Reactionary Dentine Secretion

To investigate the effect of modulation of Wnt signaling on reactionary dentine formation, a reproducible tooth damage model was established. 6 weeks old, Axin2^{-CreERT2; Rosa26-mTmG (fl/+)} and GPR₁₇₇₇(Wntless)^{-pCAGCreERT2 (fl/fl)} mice were injected intraperitoneally with three doses of tamoxifen (2 mg per 30 g mouse, SIGMA), one dose a day. 5 days after the last tamoxifen injection, the height of the middle cusp of mouse maxillary first molars were reduced without exposing the dental pulp, leaving a band of dentine to protect the inner pulp tissue (FIG. 5). Specifically, the mice were anaesthetized with a solution made with Hypnorm (Fentanyl/fluanisone—VetaPharma Ltd.), sterile water and Hypnovel (Midazolam—Roche) in the ratio 1:2:1 at the rate of 10 ml/kg intraperitonially. A rounded carbide burr FG ¼ coupled to a high-speed hand piece was used to expose the dentine of the mouse superior first molars (left and right side).

The exposed dentine was capped either with calcium hydroxide (Dycal; Dentsply) or mineral trioxide aggregate (MTA) (ProRoot MTA; Dentsply), or dry collagen sponge (Kolspon-Fish Collage Type 1; Eucare Ltd), or collagen sponge soaked in dimethyl sulfoxide (DMSO; SIGMA), or 50 nM Tideglusib, or 1 μM LY2157299, or 1 μM Dorsomorphin. All drugs were dissolved and diluted in DMSO. A layer of glass ionomer cement (Ketac-Cem Radiopaque; 3M ESPE) was used as a sealing material. Vetergesic (Buprenorphine—Ceva) was injected to all mice post-operative at the rate of 0.3 mg/kg by intraperitoneal injection as analgesic. The animals were sacrificed after 1 day and 4 weeks. A total of 14 genetically-modified mice (28 damaged molars) and 26 CD1 mice (52 molars) were used.

CD1 (wild type) were used as control and to study the effect of small molecules. Mice were collected 1 day and 4 weeks after injury.

Mice upper molars were dissected, fixed in 4% paraformaldehyde (PFA) for 24-hours at 4° C. and scanned using a Bruker Skyscan1272 micro-CT (pCT) scanner. Microview software program (GE) was used for visualization and analysis. Two-dimensional (2D) images were obtained from pCT cross-sectional images of superior first molar, to evaluate mineral formation. Three-dimensional (3D) reconstructions were used to verify pulp exposure. The dentine thickness was measured using the “Line” function of the software. For the dentine thickness-analysis the distance between the center of the roof of the middle pulp horn and the floor of the injured dentine margin was measured. In order to assess tissue mineral content a region of interest (ROI) of X=0.2 mm, Y=0.4 mm, and Z=0.2 mm was set as standard for all the samples and the mineral analysis was performed. The region measured comprised only the site of injury. ROI complete filled with mineral=0.0017 mg.

The model was first tested with the current standard materials used in dentistry, (glass ionomer, MTA, and calcium hydroxide) and showed the formation of tubular reactionary dentine and preservation of tooth vitality. The effect of modulation of Wnt/β-catenin signaling activity on reactionary dentine formation was studied using Axin2^{-LacZ/LacZ} and Wntless^{cko/cko} mice.

After 4 weeks decalcification in 19% EDTA pH 6, the teeth were embedded in wax blocks and sectioned at 8 μm thickness. Sections were histologically stained using Masson's Trichrome. The histology revealed that inhibition of Wnt activity did not prevent reactionary dentine formation or affect its tubular structure, while enhanced Wnt activity lead to a large increase in the amount of reactionary dentine formed that was disorganized and lacked a regular tubular structure (FIG. 6).

### EXAMPLE 6

### Effect of BMP and TGF-β Inhibition on Repair

Sequestered latent BMP and TGF-β proteins present in the dentine matrix have been implicated in tertiary dentine formation following damage, mainly based on results obtained from in vitro experiments. The effect of inhibition of these signalling pathways in was investigated in the in vivo model of reactionary dentine formation described in Example 5 by utilising small molecules to inhibit these signalling pathways. The small molecule LY2157299 is a TGF-β type I receptor kinase inhibitor and the small molecule Dorsomorphin is an inhibitor of BMP type I receptors ALK2, ALK3 and ALK6 (Bhola et al. 2013, Yu et al. 2008). Both compounds were first tested for cytotoxicity and effectiveness of signalling pathway blocking in vitro using 17IA4 cells. The first upper molars of CD1 mice were damaged to stimulate reactionary dentine formation and a collagen sponge was soaked in either 1 μM LY2157299 or 1 μM Dorsomorphin was used as a delivery vehicle. The sponges were placed on the exposed dentine and sealed with a layer of glass ionomer.

4 weeks after injury (FIG. 7A), the molars treated with 1 μM LY2157299 showed secretion of disorganised (globular) dentine (FIG. 7B) rather than tubular reactionary dentine observed in controls. Molars treated with 1 μM Dorsomorphin secreted a mixture of tubular dentine and globular dentine (FIG. 7C). TGF-β and BMP signalling following dentine damage appear not to be essential for reactionary dentine formation but are required for modulation of dentine structure.

### EXAMPLE 7

### Cells under the Injury Site Can Respond to Wnt/β-Catenin Signaling

Since Wnt/β-catenin signaling is required for reparative dentine formation we investigated if this pathway plays a role in reactionary dentine formation. The small molecule GSK3 antagonist Tideglusib was delivered on collagen sponges at the site of damage and sealed with glass ionomer. Sponge alone and MTA sealed with glass ionomer were used as controls. To identify whether cells responded to the drug, TCF/Lef:H2B-GFP, Axin2^{-CreERT2; Rosa26-mTmG flox/+} and CD1 wild type mice were used. TCF/Lef:H2B-GFP reporter mice allowed the visualisation of Wnt active cells (ie. Cells receiving a Wnt signal), Axin2^{-CreERT2; Rosa26-mTmG flox/+} mice were used to lineage trace Axin2-expressing cells and gene expression analysis via qPCR (as described in Example 1) was performed on pulp cells from CD1 mouse molars. The first molars of genetically modified mice were collected 1 day after the injury and immunohistochemistry was performed. Deparaffinised sections were retrieved with sodium citrate (pH 6) and incubated with chicken polyclonal anti-GFP antibody (1:500; Abcam, Cambridge, Mass., USA; ab13970) overnight at 4° C. Sections were washed and exposed to appropriate biotinylated secondary antibody, then horseradish peroxidase (HRP)-conjugated streptavidin-biotin antibody and washed with PBST. Immunoreactivity was visualized with MenaPath green chromogen kit (Bio SB). For immunofluorescence, chicken polyclonal anti-GFP antibody (1:1000; Abcam, Cambridge, Mass., USA; ab13970) was added overnight at 4° C. Sections were washed and exposed to secondary antibody (1:500; Thermo Fisher Scientific, Eugene, Oreg., USA; A21449) for 1 hour at room temperature.

Localisation of GFP showed that in TCF/Lef:H2B-GFP reporter mice, odontoblasts and pulp cells under the injury site were responsive to Wnt signalling and an increased local response to Wnt signalling at the injured pulp horn site could be observed with addition of Tideglusib (FIG. 8A,B). Axin2^{-CreERT2; Rosa26-mTmGflox/+} mice presented a similar pattern of Wnt responsiveness with more GFP-positive cells in the dental pulp when 50 nM Tideglusib was applied compared to the collagen sponge only (FIG. 8 C,D).

To confirm the elevated Axin2 gene expression in the dental pulp, P21 CD1 molars were damaged and the dental pulp collected and dissociated for qPCR analysis as described in Example 1.

The results are shown in FIG. 8E. Axin2 expression was 2-fold higher in teeth treated with Tideglusib compared to controls. Notably MTA and collagen sponge showed no effect on early-response Axin2 expression suggesting that current treatment protocols for indirect pulp capping material do not act through this pathway. These results showed that small molecule drugs such as Tideglusib are able to penetrate damaged dentine and exert effects on odontoblasts and pulp cells.

### EXAMPLE 8

### GSK-3 inhibitor Small Molecules Increase Local Reactionary Secretion

Having confirmed that Tideglusib can reach to the inner pulp through the remaining dentine band and activate Wnt signaling in odontoblasts and cells, its capacity to modulate reactionary dentine formation was evaluated using the model described in Example 5. Mouse upper first molars were damaged and capped with sponges soaked in 50 nM Tideglusib and left for 4 weeks. Histology of the upper first molars revealed that teeth indirectly capped with 50 nM Tideglusib showed enhanced reactionary dentine formation compared with controls (FIG. 9 A,A′-C,C′). Importantly, histology also showed normal tubular reactionary dentine formation with the Wnt activator and the dental pulp remained vital (FIG. 9B″,C″). Moreover, μQCT scanning confirmed by mineral content analysis an increase of mature mineral formation under the injury site, when teeth were treated with Tideglusib in comparison to collagen sponge alone (FIG. 9D). In addition, linear measurement analysis revealed that upper first molars treated with the drug presented a thicker mineral band at the site of injury than control molars with the collagen sponge alone (no drug). Compared to non-injured molars, GSK-3 antagonist treated molars showed a similar dentine thickness (FIG. 9E).

1-14. (canceled)

15. A method for repairing or regenerating dentine which comprises administering to a patient in need thereof, a pharmaceutically acceptable small molecule which inhibits GSK-3 activity.

16. The method according to claim 15 wherein the small molecule is applied topically to an area of exposed dentine.

17. The method according to claim 16 wherein the small molecule is administered in association with a matrix material.

18. The method according to claim 17 wherein the matrix material comprises a collagen sponge, which has been impregnated with the small molecule.

19. The method according to claim 17 wherein the matrix material is shaped to fill a cavity in which dentine is exposed.

20. The method according to claim 17 wherein the matrix material is held in place by means of a cap, crown or ionomer.

21. (canceled)

22. The method according to claim 15, which is a method for the treatment of dental caries or for the treatment of dental trauma.

23. The method according to claim 15, wherein the pharmaceutically acceptable small molecule is a thiadiazolidindione, or a pharmaceutically acceptable salt thereof.

24. The method according to claim 15, wherein the pharmaceutically acceptable small molecule is selected from the group consisting of formula (I): 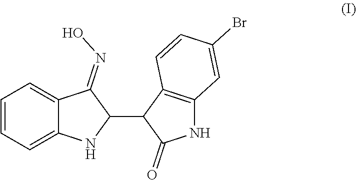 [SMILES: O=C1Nc2cc(Br)ccc2C1C1Nc2ccccc2/C1=N\O]
formula (II): 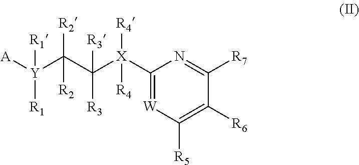
wherein:
W is optionally substituted carbon or nitrogen;
X and Y are independently selected from the group consisting of nitrogen, oxygen, and optionally substituted carbon;
A is optionally substituted aryl or heteroaryl;
R₁, R₂, R₃ and R₄ are independently selected from the group consisting of hydrogen, hydroxyl, and optionally substituted loweralkyl, cycloloweralkyl, alkylaminoalkyl, loweralkoxy, amino, alkylamino, alkylcarbonyl, arylcarbonyl, aralkylcarbonyl, heteroarylcarbonyl, heteroaralkylcarbonyl, aryl and heteroaryl;
R_{1′}, R_{2′}, R_{3′} and R_{4′} are independently selected from the group consisting of hydrogen, and optionally substituted loweralkyl;
R₆ and R₇ are independently selected from the group consisting of hydrogen, halo, and optionally substituted loweralkyl, cycloalkyl, alkoxy, amino, am inoalkoxy, alkylcarbonylamino, arylcarbonylamino, aralkylcarbonylamino, heteroarylcarbonylamino, heteroaralkylcarbonylamino, cycloimido, heterocycloimido, am idino, cycloamidino, heterocycloamidino, guanidinyl, aryl, biaryl, heteroaryl, heterobiaryl, heterocycloalkyl, and arylsulfonamido; and
R₆ is selected from the group consisting of hydrogen, hydroxy, halo, carboxyl, nitro, amino, amido, amidino, imido, cyano, and substituted or unsubstituted loweralkyl, loweralkoxy, alkylcarbonyl, arylcarbonyl, aralkylcarbonyl, heteroarylcarbonyl, heteroaralkylcarbonyl, alkylcarbonyloxy, arylcarbonyloxy, aralkylcarbonyloxy, heteroarylcarbonyloxy, heteroaralkylcarbonyloxy, alkylaminocarbonyloxy, arylaminocarbonyloxy, formyl, loweralkylcarbonyl, loweralkoxycarbonyl, am inocarbonyl, am inoaryl, alkylsulfonyl, sulfonamido, am inoalkoxy, alkylamino, heteroarylamino, alkylcarbonylamino, alkylaminocarbonylamino, arylaminocarbonylamino, aralkylcarbonylamino, heteroarylcarbonylamino, arylcarbonylamino, heteroarylcarbonylamino cycloamido, cyclothioamido, cycloamidino, heterocycloamidino, cycloimido, heterocycloimido, guanidinyl, aryl, heteroaryl, heterocyclo, heterocycloalkyl, arylsulfonyl and arylsulfonamido;
and
formula (III): 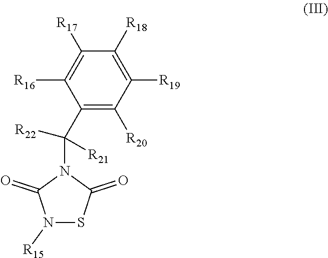
wherein
R₁₅ is an organic group having at least 8 atoms selected from C or O, which is not linked directly to the N through a —C(O)— and comprising at least an aromatic ring; and
R₁₆, R₁₇, R₁₈, R₁₉, R₂₀, R₂₁ and R₂₂ are independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, —COR₂₃, —C(O)OR₂₃, —C(O)NR₂₃R₂₄ —C—NR₂₃, —CN, —OR₂₃, —OC(O)R₂₃, —S(O)ₜ—R₂₃, —NR₂₃R₂₄, —NR₂₃C(O)R₂₄, —NO₂, —N—CR₂₃R₂₄ or halogen;
t is 0, 1, 2 or 3;
R₂₃ and R₂₄ are each independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, substituted or unsubstituted alkoxy, substituted or unsubstituted aryloxy, halogen;
wherein R₂₁ and R₂₂ together can form a group ═O, and wherein any pair R₂₁ R₁₆, R₁₆ R₁₇, R₁₇ R₁₈, R₁₈ R₁₉, R₁₉ R₂₀, R₂₀ R₂₂, or R₂₃ R₂₄ can form together a cyclic substituent;
or a pharmaceutically acceptable salt thereof.

25. The method according to claim 24, wherein the pharmaceutically acceptable small molecule is BIO (6-bromoindirubin-3′-oxime), CHIR₉₉₀₂₁ (6-[[2-[[4-(2,4-Dichlorophenyl)-5-(5-methyl-1H-imidazol-2-yl)-2- pyrimidinyl]amino]ethyl]amino]-3-pyridinecarbonitrile), or tideglusib (4-benzyl-2-(naphthalen-1-yl)-[1,2,4]thiadiazolidine-3,5-dione).

26. A combination of a matrix material suitable for use in a dental implant, and a pharmaceutically acceptable small molecule which inhibits GSK-3 activity.

27. The combination according to claim 26, wherein the matrix material is biodegradable.

28. The combination according to claim 27, wherein the matrix material is porous.

29. The combination according to claim 28, wherein the pharmaceutically acceptable small molecule is impregnated into the matrix material.

30. The combination according to claim 28, wherein the matrix material is a collagen sponge.

31. The combination according to claim 26, wherein the pharmaceutically acceptable small molecule is a thiadiazolidindione, or a pharmaceutically acceptable salt thereof.

32. The combination according to claim 26, wherein the pharmaceutically acceptable small molecule is selected from the group consisting of formula (I): 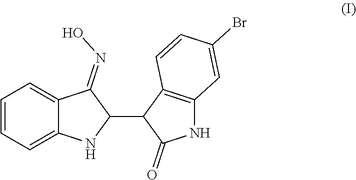 [SMILES: O=C1Nc2cc(Br)ccc2C1C1Nc2ccccc2/C1=N\O]
formula (II): 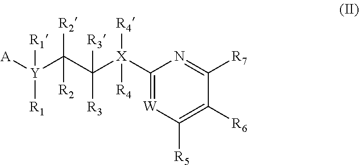
wherein:
W is optionally substituted carbon or nitrogen;
X and Y are independently selected from the group consisting of nitrogen, oxygen, and optionally substituted carbon;
A is optionally substituted aryl or heteroaryl;
R₁, R₂, R₃ and R₄ are independently selected from the group consisting of hydrogen, hydroxyl, and optionally substituted loweralkyl, cycloloweralkyl, alkylaminoalkyl, loweralkoxy, amino, alkylamino, alkylcarbonyl, arylcarbonyl, aralkylcarbonyl, heteroarylcarbonyl, heteroaralkylcarbonyl, aryl and heteroaryl;
R_{1′}, R_{2′}, R_{3′}, and R_{4′}, are independently selected from the group consisting of hydrogen, and optionally substituted loweralkyl;
R₆ and R₇ are independently selected from the group consisting of hydrogen, halo, and optionally substituted loweralkyl, cycloalkyl, alkoxy, amino, aminoalkoxy, alkylcarbonylamino, arylcarbonylamino, aralkylcarbonylamino, heteroarylcarbonylamino, heteroaralkylcarbonylamino, cycloimido, heterocycloimido, amidino, cycloamidino, heterocycloamidino, guanidinyl, aryl, biaryl, heteroaryl, heterobiaryl, heterocycloalkyl, and arylsulfonamido; and
R₆ is selected from the group consisting of hydrogen, hydroxy, halo, carboxyl, nitro, amino, amido, amidino, imido, cyano, and substituted or unsubstituted loweralkyl, loweralkoxy, alkylcarbonyl, arylcarbonyl, aralkylcarbonyl, heteroarylcarbonyl, heteroaralkylcarbonyl, alkylcarbonyloxy, arylcarbonyloxy, aralkylcarbonyloxy, heteroarylcarbonyloxy, heteroaralkylcarbonyloxy, alkylaminocarbonyloxy, arylaminocarbonyloxy, formyl, loweralkylcarbonyl, loweralkoxycarbonyl, am inocarbonyl, am inoaryl, alkylsulfonyl, sulfonamido, am inoalkoxy, alkylamino, heteroarylamino, alkylcarbonylamino, alkylaminocarbonylamino, arylaminocarbonylamino, aralkylcarbonylamino, heteroarylcarbonylamino, arylcarbonylamino, heteroarylcarbonylamino cycloamido, cyclothioamido, cycloamidino, heterocycloamidino, cycloimido, heterocycloimido, guanidinyl, aryl, heteroaryl, heterocyclo, heterocycloalkyl, arylsulfonyl and arylsulfonamido;
and
formula (III): 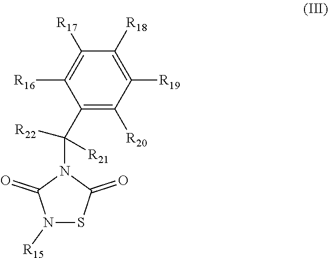
wherein
R₁₅ is an organic group having at least 8 atoms selected from C or O, which is not linked directly to the N through a —C(O)— and comprising at least an aromatic ring; and
R₁₆, R₁₇, R₁₈, R₁₉, R₂₀, R₂₁ and R₂₂ are independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, —COR₂₃, —C(O)OR₂₃, —C(O)NR₂₃R₂₄ —C—NR₂₃, —CN, —OR₂₃, —OC(O)R₂₃, —S(O)ₜ—R₂₃, —NR₂₃R₂₄, —NR₂₃C(O)R₂₄, —NO₂, —N—CR₂₃R₂₄ or halogen;
t is 0, 1, 2 or 3;
R₂₃ and R₂₄ are each independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, substituted or unsubstituted alkoxy, substituted or unsubstituted aryloxy, halogen;
wherein R₂₁ and R₂₂ together can form a group ═O, and wherein any pair R₂₁ R₁₆, R₁₆ R₁₇, R₁₇ R₁₈, R₁₈ R₁₉, R₁₉ R₂₀, R₂₀ R₂₂, or R₂₃ R₂₄ can form together a cyclic substituent;
or a pharmaceutically acceptable salt thereof.

33. The combination according to claim 32, wherein the pharmaceutically acceptable small molecule is BIO (6-bromoindirubin-3′-oxime), CHIR₉₉₀₂₁ (6-[[2-[[4-(2,4-Dichlorophenyl)-5-(5-methyl-1H-imidazol-2-yl)-2-pyrim idinyl]amino]ethyl]amino]-3-pyridinecarbonitrile), or tideglusib (4-benzyl-2-(naphthalen-1-yl)-[1,2,4]thiadiazolidine-3,5-dione).

34. The combination according to claim 26, which further comprises an antibiotic, a transforming growth factor beta (TGF-β) agonist, a bone morphogenetic protein (BMP) agonist, or a combination thereof.

35. A kit comprising the combination according to claim 26.


SEQUENCE LISTING (4 sequences)

SEQ ID NO: 1 - DNA, 20 bp, Artificial Sequence
  primer
ggctgtattcccctccatcg

SEQ ID NO: 2 - DNA, 21 bp, Artificial Sequence
  Primer
ccagttggtaacaatgcctgt

SEQ ID NO: 3 - DNA, 21 bp, Artificial Sequence
  primer
tgactctccttccagatccca

SEQ ID NO: 4 - DNA, 19 bp, Artificial Sequence
  primer
tgcccacactaggctgaca